(19) United States
(12) Patent Application Publication
(10) Pub. No.: US 2012/0232264 A1
(43) Pub. Date: Sep. 13, 2012

(54) SYSTEM AND METHOD FOR TREATING BIOMASS

(76) Inventors: Kenji Sato, Fujisawa-shi (JP); Kentaro Nariai, Yokohama-shi (JP); Makoto Kitano, Tokyo (JP); Norimitsu Kaneko, Yokohama-shi (JP); Michikazu Hara, Yokohama-shi (JP); Daizo Yamaguchi, Tsuyama-shi (JP)

(21) Appl. No.: 13/497,983

(22) PCT Filed: Sep. 24, 2010
(86) PCT No.: PCT/JP2010/066575

§ 371 Date: May 31, 2012

(30) Foreign Application Priority Data
Sep. 24, 2009 (JP) 2009-219362

(51) Int. Cl. C07H 1/00; B01J 10/00

(52) U.S. Cl. 536/124; 422/162

(57) ABSTRACT

A biomass treatment apparatus (A) is constituted by a pressurized hot water reaction apparatus (1) in which pressurized hot water is used, biomass is hydrolyzed under first reaction conditions that provide for decomposition of hemicellulose so as to generate a first polysaccharide solution including xylooligosaccharides, and then the biomass is hydrolyzed under second reaction conditions that provide for decomposition of cellulose to generate a second polysaccharide solution including cellooligosaccharides, a first catalysis apparatus (2) in which the first polysaccharide solution that flows out from the pressurized hot water reaction apparatus (1) is hydrolyzed using a solid acid catalyst so as to generate a first monosaccharide solution including xylose, and a second catalysis apparatus (3) in which the second polysaccharide solution that flows out from the pressurized hot water reaction apparatus (1) is hydrolyzed using a solid acid catalyst so as to generate a second monosaccharide solution including glucose.

### TECHNICAL FIELD

The present invention relates to biomass treatment apparatus and method.

Priority is claimed on Japanese Patent Application No. 2009-219362, filed Sep. 24, 2009, the content of which is incorporated herein by reference.

### BACKGROUND ART

Recently, biomass, which is a carbon neutral resource, is receiving attention as an alternative resource for fossil resources, such as petroleum. As one use of the biomass, a technique is known in which biomass containing cellulose is subjected to a saccharification treatment so as to generate glucose (monosaccharide), and the glucose undergoes alcohol fermentation, thereby generating bioethanol.

As a method of generating glucose from biomass (saccharification technique), a method in which biomass is hydrolyzed by addition of sulfuric acid (the dilute sulfuric acid method, the concentrated sulfuric acid method, and the like) is generally known. However, in this method, there is a problem of corrosion of the reactor, the necessity of liquid waste disposal, impossibility of recycling due to the resultant liquid phase, or the like. In addition, a method in which saccharification is carried out using an enzyme (refer to the following Patent Document 1) is also known. However, there is a problem with this method in that enzyme is expensive and unrecyclable, which results in an increase in the manufacturing costs.

In consideration of the above problems, a method in which biomass is saccharified using a solid acid catalyst that has an equivalent catalyst performance to that of sulfuric acid has no problem of corrosion of a reactor and liquid waste disposal, and can be recycled at low cost (for example, a so-called solid sulfuric acid having a sulfo group supported by carbon, zeolite, or the like) (refer to the following Non-Patent Document 1) is also proposed. However, even in this method, there are problems in that the solids react with each other such that the reaction rate becomes extremely slow, and it becomes difficult to separate residue and the solid acid catalyst.

In addition, a method in which biomass is hydrolyzed using supercritical water or subcritical water, thereby generating glucose (refer to the following Patent Document 2) is also proposed. However, there is a problem with this method in that, since glucose is decomposed more easily than cellulose, glucose is generated and decomposed at the same time, and, consequently, the saccharide yield is degraded. In addition, in a case in which biomass includes hemicellulose, since hemicellulose has a lower decomposition temperature than cellulose, there is a problem in that hemicellulose is decomposed before the decomposition temperature of cellulose is reached, and, consequently, an ethanol fermentation inhibitor (for example, furfural) is generated.

In consideration of the above problems, a method in which pressurized hot water in a subcritical state is made to flow through a saccharification vessel filled with cellulose powder, and hydrolysis is carried out, thereby generating a water-insoluble polysaccharide is proposed (refer to the following Patent Document 2). Patent Document 2 describes that generation of an ethanol fermentation inhibitor is suppressed by setting the temperature of the pressurized hot water that is made to flow through the saccharification vessel to 240° C. to 340° C. (preferably 270° C. to 310° C.), and the saccharide yield is improved. However, there are problems with the technique of Patent Document 2 in that the temperature of the pressurized hot water is set only for the saccharification subject of cellulose, saccharification of hemicellulose is not taken into account, and the saccharide yield is not further improved.

### CITATION LIST

### Patent Document

[Patent Document 1]: Japanese Unexamined Patent Application, Publication No. 2006-136263

[Patent Document 2]: Japanese Patent No. 3128575

### Non-Patent Document

[Non-Patent Document 1]: Seminar text “The pretreatment and saccharification of biomass oriented for the manufacture of ethanol fuels” (Technical Information Institute Co., Ltd.) (2009)

[Non-Patent Document 2]: Katsunobu Ehara, Shirou Saka, HORIBA Technical Report, 31, 98-105 (2005)

### SUMMARY OF INVENTION

### Technical Problem

As described above, in the past, a method in which a solid acid catalyst is used and a method in which pressurized hot water is used were proposed as promising biomass saccharification techniques. However, both methods still have problems to be solved, and there is a demand for rapid development of countermeasure techniques for handling global warming and creating a recycling society in the future.

The invention has been made in consideration of the above circumstances, and an object of the invention is to provide a biomass treatment apparatus and a method which can further improve the saccharide yield, the performance of a solid acid catalyst (specifically, improvement of the reaction rate), and ease of isolation.

### Solution to Problem

A first aspect of the biomass treatment apparatus according to the invention has a pressurized hot water reaction apparatus in which pressurized hot water is used, biomass is hydrolyzed under first reaction conditions that provide for decomposition of hemicellulose so as to generate a first polysaccharide solution including xylooligosaccharides, and then the biomass is hydrolyzed under second reaction conditions that provide for decomposition of cellulose so as to generate a second polysaccharide solution including cellooligosaccharides; a first catalysis apparatus in which the first polysaccharide solution that flows out from the pressurized hot water reaction apparatus is hydrolyzed using a solid acid catalyst so as to generate a first monosaccharide solution including xylose; and a second catalysis apparatus in which the second polysaccharide solution that flows out from the pressurized hot water reaction apparatus is hydrolyzed using a solid acid catalyst so as to generate a second monosaccharide solution including glucose.

In a second aspect of the biomass treatment apparatus of the invention according to the first aspect, the first reaction conditions and the second reaction conditions are set by combining the ratio of the supply amount of the pressurized hot water and the supply amount of the biomass and the temperature of the pressurized hot water.

In a third aspect of the biomass treatment apparatus of the invention according to the first or second aspect, the first catalysis apparatus has a first mixing apparatus in which the first polysaccharide solution that flows out from the pressurized hot water reaction apparatus and the solid acid catalyst are mixed so as to generate the xylose, and a first solid and liquid separating apparatus that separates the xylose that flows out from the first mixing apparatus and a first liquid mixture including the solid acid catalyst into solids and liquids so as to separate the first monosaccharide solution including the xylose and the solid acid catalyst, and, consequently, collects and supplies the solid acid catalyst to the first mixing apparatus.

In a fourth aspect of the biomass treatment apparatus of the invention according to the first aspect, the second catalysis apparatus has a second mixing apparatus in which the second polysaccharide solution that flows out from the pressurized hot water reaction apparatus and the solid acid catalyst are mixed so as to generate the glucose, and a second solid and liquid separating apparatus that separates the glucose that flows out from the second mixing apparatus and a second liquid mixture including the solid acid catalyst into solid and liquid so as to separate the second monosaccharide solution including the glucose and the solid acid catalyst, and, consequently, collects and supplies the solid acid catalyst to the second mixing apparatus.

In a fifth aspect of the biomass treatment apparatus of the invention according to the first aspect, a first fermentation apparatus in which the first monosaccharide solution generated by the first catalysis apparatus undergoes alcohol fermentation so as to generate bioethanol is provided.

In a sixth aspect of the biomass treatment apparatus of the invention according to the first aspect, a second fermentation apparatus in which the second monosaccharide solution generated by the second catalysis apparatus undergoes alcohol fermentation so as to generate bioethanol is provided.

A first aspect of the biomass treatment method according to the invention has a pressurized hot water reaction process in which pressurized hot water is used, biomass is hydrolyzed under first reaction conditions that provide for decomposition of hemicellulose so as to generate a first polysaccharide solution including xylooligosaccharides, and then the biomass is hydrolyzed under second reaction conditions that provide for decomposition of cellulose so as to generate a second polysaccharide solution including cellooligosaccharides; a first catalyst reaction process in which the first polysaccharide solution generated by the pressurized hot water reaction process is hydrolyzed using a solid acid catalyst so as to generate a first monosaccharide solution including xylose; and a second catalyst reaction process in which the second polysaccharide solution generated by the pressurized hot water reaction process is hydrolyzed using a solid acid catalyst so as to generate a second monosaccharide solution including glucose.

### Advantageous Effects of Invention

According to the biomass treatment apparatus and the treatment method according to the invention, decomposed substances of hemicellulose (polysaccharides including xylooligosaccharides) and decomposed substances of cellulose (polysaccharides including cellooligosaccharides) can be separately selected by dividing and controlling reaction conditions that provide for hydrolysis of biomass in the pressurized hot water reaction apparatus into two steps of first reaction conditions that provide for decomposition of hemicellulose and second reaction conditions that provide for decomposition of cellulose. As a result, the amount of a final resultant of monosaccharides that can undergo alcohol fermentation (glucose and xylose) can be increased, and the saccharide yield can be improved.

In addition, since the contact reactions between the polysaccharide solutions (the first polysaccharide solution and the second saccharide solution) and the solid acid catalyst in the first catalysis apparatus and the second catalysis apparatus are solid-liquid reactions, the reaction rate is not slow (the reaction rate is high), and the solid acid catalyst can be easily separated and collected.

In addition, since the number of the pressurized hot water reaction apparatuses that supply polysaccharides to the first catalysis apparatus and the second catalysis apparatus is merely one, the capacity of the biomass treatment apparatus can be decreased. That is, a compact and cheap biomass treatment apparatus can be provided.

Furthermore, since all treatment subjects that flow in and out between the respective treatment processes are configured to move in a liquid state, the treatment can be carried out more efficiently than treatments in which treatment subjects move in a solid state. In addition, it is possible to decrease the number of pipes for moving solids in the biomass treatment apparatus. That is, a more compact and cheaper biomass treatment apparatus can be provided.

Furthermore, a hydrothermal reaction by pressurized hot water and a solid acid catalyst reaction are carried out in a stepwise manner, generation of substances that inhibit ethanol fermentation is suppressed, and the saccharide yield can be further improved.

### BRIEF DESCRIPTION OF THE DRAWINGS

FIG. 1 is a block diagram of a biomass treatment apparatus A according to a first embodiment of the invention.

FIG. 2 is a modified example of a first catalysis apparatus 2 (a second catalysis apparatus 3) in the biomass treatment apparatus A.

### DESCRIPTION OF EMBODIMENTS

Hereinafter, an embodiment of the invention will be described with reference to the accompanying drawings.

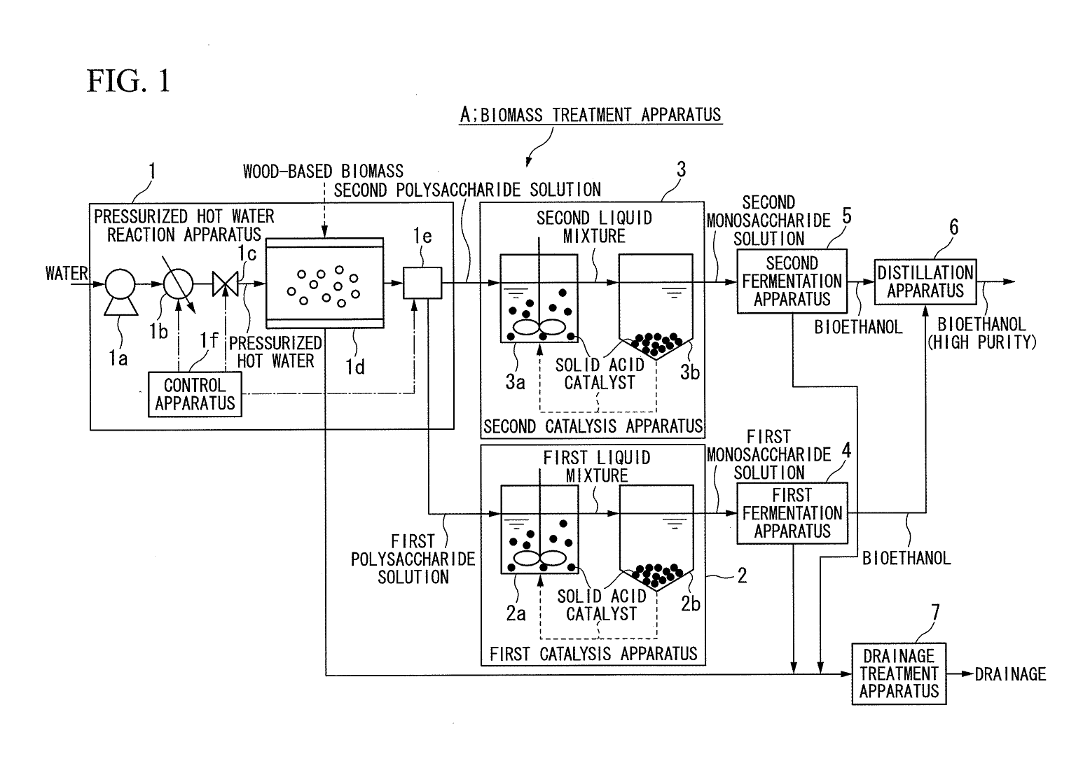

FIG. 1 is a schematic configuration view of a biomass treatment apparatus A in the present embodiment. As shown in FIG. 1, the biomass treatment apparatus A is a chemical plant that generates bioethanol using wood-based biomass containing cellulose, hemicellulose, and lignin as the main components, such as wood chips or construction waste, which are biomass, as a raw material. The biomass treatment apparatus A is constituted by a pressurized hot water reaction apparatus 1, a first catalysis apparatus 2, a second catalysis apparatus 3, a first fermentation apparatus 4, a second fermentation apparatus 5, a distillation apparatus 6, and a drainage treatment apparatus 7.

Meanwhile, in FIG. 1, the solid line indicates a flow of liquid, the broken line indicates a flow of solids, and the dotted line indicates a flow of an electric signal.

The cellulose included in the wood-based biomass refers to a polysaccharide represented by a chemical formula H(C₆H₁₀O₅)ₙOH as is well-known, and is a high-molecular-weight compound that is polymerized glucose (a monosaccharide) including six carbon atoms. In addition, the hemicellulose is a high-molecular-weight compound that is polymerized monosaccharides other than glucose (mainly monosaccharides including five carbon atoms). In addition, lignin refers to a high-molecular-weight compound that has a three-dimensional grain structure, and forms woody material by surrounding the cellulose and the hemicellulose. Since lignin is a substance that inhibits decomposition of cellulose and hemicellulose, it is preferable to remove lignin from wood-based biomass through a pretreatment, and supply the pretreated wood-based biomass to the pressurized hot water reaction apparatus 1.

The pressurized hot water reaction apparatus 1 is, for example, a hot water circulation-type reaction apparatus. In the pressurized hot water reaction apparatus 1, pressurized hot water is used, wood-based biomass is hydrolyzed under first reaction conditions that provide for decomposition of hemicellulose so as to generate a first polysaccharide solution including xylooligosaccharides, and then the wood-based biomass is subsequently hydrolyzed under second reaction conditions that provide for decomposition of cellulose so as to generate a second polysaccharide solution including cellooligosaccharides. Meanwhile, “pressurized hot water” refers to hot water in a subcritical state, and hot water pressurized to maintain the liquid state.

In addition, the pressurized hot water reaction apparatus 1 is constituted by a pump 1a, a heater 1b, a water amount adjusting valve 1c, a reaction tank 1d, a splitter 1e, and a control apparatus 1f. The pump 1a pressurizes water supplied from the outside, and sends the water to the heater 1b. The heater 1b heats the pressurized water that flows in from the pump 1a to a predetermined temperature in accordance with a temperature control signal that is input from the control apparatus 1f, and sends the pressurized hot water to the water amount adjusting valve 1c. The water amount adjusting valve 1c is an electronic control valve whose aperture is adjusted in accordance with a flow amount control signal that is input from the control apparatus 1f, which adjusts the flow amount of the pressurized hot water that flows in from the heater 1b, and then sends the flow-amount-adjusted pressurized hot water to the reaction tank 1d.

The reaction tank 1d is a vessel the internal space of which is filled with a predetermined amount of the wood-based biomass supplied from the outside, and is configured to have the pressurized hot water that flows in from the flow amount adjusting valve 1c flow through the internal space filled with the wood-based biomass and then flow out to the splitter 1e at the rear end. Therefore, the pressurized hot water and the wood-based biomass are brought into contact with each other by continuously flowing the pressurized hot water through the reaction tank 1d, and the hydrolysis of the wood-based biomass is promoted. The pressurized hot water that flows out from the reaction tank 1d to the splitter 1e includes hydrolyzed substances generated by the hydrolysis of the wood-based biomass (the polysaccharides as described below). Hereinafter, such pressurized hot water including polysaccharides is called a polysaccharide solution.

The splitter 1e selectively sends the pressurized hot water that flows in from the reaction tank 1d (the polysaccharide solution) to either of the first catalysis apparatus 2 and the second catalysis apparatus 3 in accordance with a splitting control signal that is input from the control apparatus 1f. Meanwhile, since the polysaccharide solution that flows out from the reaction tank 1d has a high temperature, it is preferable to cool the polysaccharide solution and then flow the polysaccharide solution in the splitter 1e.

The control apparatus 1f has a function of outputting temperature control signals to the heater 1b and flow amount control signals to the water amount adjusting valve 1c, and controlling the temperature and flow amount (supply amount) of pressurized hot water that is to be supplied to the reaction tank 1d, thereby selectively switching the first reaction conditions that provide for decomposition of hemicellulose and the second reaction conditions that provide for decomposition of cellulose. Here, the first reaction conditions are conditions that are used to decompose hemicellulose included in wood-based biomass and generate polysaccharides including xylooligosaccharides as the main component (hemicellulose fraction), and the second reaction conditions are conditions that are used to decompose cellulose included in wood-based biomass and generate polysaccharides including cellooligosaccharides as the main component (cellulose fraction).

The first reaction conditions and the second reaction conditions are set by combining the ratio K(=Q/V) of the supply amount Q (ml) of pressurized hot water to the supply amount V (g) of wood-based biomass and the temperature T (° C.) of pressurized hot water. The supply amount V of wood-based biomass is an unambiguously determined value that depends on the capacity of the reaction tank 1d.

For example, the temperature T of pressurized hot water may be 100° C. to less than 200° C. in the first reaction conditions and 200° C. to 290° C. in the second reaction conditions. In addition, the ratio K of the supply amount Q of pressurized hot water to the supply amount V of wood-based biomass is preferably K<100 (more preferably K<20) for both the first reaction conditions and the second reaction conditions. Meanwhile, the numeric values are merely an example in a case in which wood-based biomass is used as a raw material, and can be changed in accordance with the kind of biomass that is used as a raw material, the capacity of the reaction tank 1d, or the like.

In the hot water circulation-type reaction apparatus of the related art (for example, refer to PTL 2), since reaction conditions were set by combining the temperature (240° C. to 340° C.) and residence time (30 seconds) of pressurized hot water, there were problems in that input energy was large, and reaction control was difficult. However, as described above, wasteful supply of pressurized hot water can be decreased by using the ratio K of the supply amount Q of pressurized hot water to the supply amount V of wood-based biomass instead of the residence time. As a result, input energy can be decreased, and reaction control becomes easy.

The control apparatus 1f firstly controls the temperature T and supply amount Q of pressurized hot water in the reaction tank 1d so as to cause hydrolysis of wood-based biomass under the first reaction conditions, and then controls the temperature T and supply amount Q of pressurized hot water so as to cause hydrolysis of wood-based biomass under the second reaction conditions. Therefore, the pressurized hot water that flows out from the reaction tank 1d when the first reaction conditions are used includes polysaccharides including xylooligosaccharides as the main component (hemicellulose fraction), and the pressurized hot water that flows out from the reaction tank 1d when the second reaction conditions are used includes polysaccharides including cellooligosaccharides as the main component (cellulose fraction).

Hereinafter, the pressurized hot water including polysaccharides having xylooligosaccharides as the main component will be called a first polysaccharide solution, and the pressurized hot water including polysaccharides having cellooligosaccharides as the main component will be called a second polysaccharide solution.

In addition, the control apparatus 1f controls the splitter 1e so that the pressurized hot water that flows out from the reaction tank 1d when the first reaction conditions are used (the first polysaccharide solution) is sent to the first catalysis apparatus 2. Furthermore, the control apparatus controls the splitter 1e so that the pressurized hot water that flows out from the reaction tank 1d when the second reaction conditions are used (the second polysaccharide solution) is sent to the second catalysis apparatus 3.

The first catalysis apparatus 2 hydrolyzes the first polysaccharide solution that flows out from the pressurized hot water reaction apparatus 1 (specifically the splitter 1e) when the first reaction conditions are used using a solid acid catalyst so as to generate the first polysaccharide solution including xylose. The first catalysis apparatus 2 is constituted by a first mixing apparatus 2a and a first solid-liquid separating apparatus 2b.

The first mixing apparatus 2a stirs and mixes the first polysaccharide solution that flows in from the pressurized hot water reaction apparatus 1 and a previously filled solid acid catalyst so as to bring both into contact and promote a hydrolysis reaction (that is, saccharification reaction). Therefore, the xylooligosaccharides included in the first polysaccharide solution are decomposed so as to generate xylose, which is a monosaccharide. A first liquid mixture including the generated xylose and the solid acid catalyst is flowed out from the first mixing apparatus 2a to the first solid-liquid separating apparatus 2b.

The first solid-liquid separating apparatus 2b separates the first liquid mixture that flows in from the first mixing apparatus 2a into solids and liquids so as to separate a first monosaccharide solution including xylose and the solid acid catalyst, and collects and supplies (recycles) the solid acid catalyst to the first mixing apparatus 2a. In addition, the first solid-liquid separating apparatus 2b sends the first monosaccharide solution including xylose to the first fermentation apparatus 4. A settlement tank can be used as the first solid-liquid separating apparatus 2b. That is, the solid acid catalyst that is a solid in the first liquid mixture supplied to the settlement tank settles at the tank bottom portion, and a supernatant solution obtains the first monosaccharide solution including xylose.

The second catalysis apparatus 3 hydrolyzes the second polysaccharide solution that flows out from the pressurized hot water reaction apparatus 1 (specifically the splitter 1e) when the second reaction conditions are used using a solid acid catalyst so as to generate the second polysaccharide solution including glucose. The second catalysis apparatus 3 is constituted by a second mixing apparatus 3a and a second solid-liquid separating apparatus 3b.

The second mixing apparatus 3a stirs and mixes the second polysaccharide solution that flows in from the pressurized hot water reaction apparatus 1 and a previously filled solid acid catalyst so as to bring both into contact and promote a hydrolysis reaction (that is, saccharification reaction). Therefore, the cellooligosaccharides included in the second polysaccharide solution are decomposed so as to generate glucose which is a monosaccharide. A second liquid mixture including the generated glucose and the solid acid catalyst is flowed out from the second mixing apparatus 3a to the second solid-liquid separating apparatus 3b.

The second solid-liquid separating apparatus 3b separates the second liquid mixture that flows in from the second mixing apparatus 3a into solid and liquid so as to separate a second monosaccharide solution including glucose and the solid acid catalyst, and collects and supplies (recycles) the solid acid catalyst to the second mixing apparatus 3a. In addition, the second solid-liquid separating apparatus 3b sends the second monosaccharide solution including glucose to the second fermentation apparatus 5. Similarly to the first solid-liquid separating apparatus 2b, a settlement tank can be used as the second solid-liquid separating apparatus 3b. That is, the solid acid catalyst that is a solid in the second liquid mixture supplied to the settlement tank settles at the tank bottom portion, and a supernatant solution obtains the second monosaccharide solution including glucose.

The first fermentation apparatus 4 adds an ethanol-fermentation microbe, such as yeast, and a nutrient source, such as nitrogen or phosphorous, to the first monosaccharide solution including the xylose that flows in from the first catalysis apparatus 2 (specifically the first solid-liquid separating apparatus 2b), and cultures the microbe under conditions of appropriate temperature, pH, and the like so as to carry out alcohol fermentation of the first monosaccharide solution including xylose, thereby generating bioethanol. A variety of well-known microbes, such as Saccharomyces yeast, can be used as the ethanol-fermentation microbe. The generated bioethanol is flowed out from the first fermentation apparatus 4 to the distillation apparatus 6.

The second fermentation apparatus 5 adds an ethanol-fermentation microbe, such as yeast, and a nutrient source, such as nitrogen or phosphorous, to the second monosaccharide solution including the glucose that flows in from the second catalysis apparatus 3 (specifically the second solid-liquid separating apparatus 3b), and cultures the microbe under conditions of appropriate temperature, pH, and the like so as to carry out alcohol fermentation of the second monosaccharide solution including glucose, thereby generating bioethanol. The generated bioethanol is flowed out from the second fermentation apparatus 5 to the distillation apparatus 6.

The distillation apparatus 6 distills and concentrates the bioethanol that flows in from the first fermentation apparatus 4 and the second fermentation apparatus 5 so as to generate highly pure bioethanol and send the bioethanol to the outside. In addition, the drainage treatment apparatus 7 drains blow water discharged from the reaction tank 1d of the pressurized hot water reaction apparatus 1 and water discharged from the first fermentation apparatus 4 and the second fermentation apparatus 5 (water generated in the process of alcohol fermentation) to the outside.

Next, a biomass treatment process in which the biomass treatment apparatus A is configured as described above (biomass treatment method) will be described. Meanwhile, the inside of the reaction tank 1d of the pressurized hot water reaction apparatus 1 is presumed to be filled with a predetermined amount of wood-based biomass in advance.

The control apparatus 1f of the pressurized hot water reaction apparatus 1 firstly controls the temperature T and supply amount Q of pressurized hot water in the reaction tank 1d so as to cause hydrolysis of the wood-based biomass under the first reaction conditions. Therefore, in the reaction tank 1d, pressurized hot water that is controlled to have a temperature T (100° C. to less than 200° C.) and a supply amount Q (Q satisfying K<100, preferably K<20) that satisfy the first reaction conditions, that is, conditions in which the hemicellulose included in the wood-based biomass is decomposed, and polysaccharides including xylooligosaccharides as the main component (hemicellulose fraction) are generated in the reaction tank 1d is flowed.

The hydrolysis of the wood-based biomass (particularly hemicellulose) is promoted by flowing the pressurized hot water that satisfies the first reaction conditions in the reaction tank 1d. In addition, the pressurized hot water that flows out from the reaction tank 1d includes polysaccharides having xylooligosaccharides as the main component (hemicellulose fraction). The pressurized hot water including polysaccharides having xylooligosaccharides as the main component flows in the splitter 1e as the first polysaccharide solution. At this time, the control apparatus 1f controls the splitter 1e so that the first polysaccharide solution is sent to the first catalysis apparatus 2 when the first reaction conditions are used. Therefore, the first polysaccharide solution that flows out from the reaction tank 1d is sent to the first catalysis apparatus 2 through the splitter 1e.

The first polysaccharide solution supplied to the first catalysis apparatus 2 is mixed with a solid acid catalyst by the first mixing apparatus 2a so as to promote a saccharification reaction. Therefore, the xylooligosaccharides included in the first polysaccharide solution are decomposed so as to generate xylose, which is a monosaccharide. In addition, the first liquid mixture including the xylose and the solid acid catalyst is flowed out from the first mixing apparatus 2a to the first solid-liquid separating apparatus 2b. The first liquid mixture is separated into the first saccharide solution including xylose and the solid acid catalyst using the first solid-liquid separating apparatus 2b, and the solid acid catalyst is supplied to the first mixing apparatus 2a and recycled. The first monosaccharide solution is sent to the first fermentation apparatus 4. In the first fermentation apparatus 4, the bioethanol generated by alcohol fermentation of the first monosaccharide solution is sent to the distillation apparatus 6, and highly pure bioethanol is generated.

Subsequently, the control apparatus 1f of the pressurized hot water reaction apparatus 1 controls the temperature T and supply amount Q of pressurized hot water in the reaction tank 1d so as to cause hydrolysis of the wood-based biomass under the second reaction conditions. Therefore, in the reaction tank 1d, pressurized hot water is flowed that is controlled to have a temperature T (200° C. to 290° C.) and a supply amount Q (Q satisfying K<100, preferably K<20) that satisfy the second reaction conditions, that is, conditions in which the cellulose included in the wood-based biomass is decomposed, and polysaccharides including cellooligosaccharides as the main component (cellulose fraction) are generated.

The hydrolysis of the wood-based biomass (particularly cellulose) is promoted by flowing the pressurized hot water that satisfies the second reaction conditions in the reaction tank 1d. In addition, the pressurized hot water that flows out from the reaction tank 1d includes polysaccharides having cellooligosaccharides as the main component (cellulose fraction). The pressurized hot water including polysaccharides having cellooligosaccharides as the main component flows in the splitter 1e as the second polysaccharide solution. At this time, the control apparatus 1f controls the splitter 1e so that the second polysaccharide solution is sent to the second catalysis apparatus 3 when the second reaction conditions are used. Therefore, the second polysaccharide solution that flows out from the reaction tank 1d is sent to the second catalysis apparatus 3 through the splitter 1e.

The second polysaccharide solution supplied to the second catalysis apparatus 3 is mixed with a solid acid catalyst by the second mixing apparatus 3a so as to promote a saccharification reaction. Therefore, the cellooligosaccharides included in the second polysaccharide solution are decomposed so as to generate glucose which is a monosaccharide. In addition, the second liquid mixture including the glucose and the solid acid catalyst is flowed out from the second mixing apparatus 3a to the second solid-liquid separating apparatus 3b. The second liquid mixture is separated into the second saccharide solution including glucose and the solid acid catalyst using the second solid-liquid separating apparatus 3b, and the solid acid catalyst is supplied to the first mixing apparatus 3a and recycled. In addition, the second monosaccharide solution is sent to the second fermentation apparatus 5. In the second fermentation apparatus 5, the bioethanol generated by alcohol fermentation of the second monosaccharide solution is sent to the distillation apparatus 6, and highly pure bioethanol is generated.

As described above, in the embodiment, the reaction conditions that provide for the hydrolysis of wood-based biomass in the pressurized hot water reaction apparatus 1 are divided into two steps of the first reaction conditions that provide for decomposition of hemicellulose and the second reaction conditions that provide for decomposition of cellulose, and controlled. Therefore, decomposed substances of hemicellulose (polysaccharides including xylooligosaccharides) and decomposed substances of cellulose (polysaccharides including cellooligosaccharides) can be separately selected. As a result, the amount of a final resultant of monosaccharides that can undergo alcohol fermentation (glucose and xylose) can be increased, and the saccharide yield can be improved.

In addition, in the embodiment, since the contact reactions between the polysaccharide solutions (the first polysaccharide solution and the second saccharide solution) and the solid acid catalyst in the first catalysis apparatus 2 and the second catalysis apparatus 3 are solid-liquid reactions, the reaction rate is slow. In addition, the solid acid catalyst can be easily separated and collected.

Furthermore, a hydrothermal reaction and a solid acid catalyst reaction are carried out in a stepwise manner, generation of a substance that inhibits ethanol fermentation is suppressed, and the saccharide yield can be further improved.

In addition, since all the polysaccharides (the first polysaccharides and the second polysaccharides) and the monosaccharides (the first monosaccharides and the second monosaccharides) in the pressurized hot water reaction apparatus 1, the first catalysis apparatus 2, and the second catalysis apparatus 3 flow in and out from the respective apparatuses in a liquid state, the treatment can be carried out more efficiently than treatments in which the polysaccharides and the monosaccharides flow in and out in a solid state. In addition, it is possible to decrease the number of pipes for moving solids in the apparatus. That is, a compact and cheap apparatus can be provided.

Meanwhile, the invention is not limited to the embodiment, and the following modified examples can be considered.

(1) In the embodiment, the biomass treatment apparatus A that generates bioethanol from wood-based biomass is exemplified, but an embodiment may be an apparatus that does not include the first fermentation apparatus 4, the second fermentation apparatus 5, and the distillation apparatus 6, and generates monosaccharides (glucose and xylose) that can undergo alcohol fermentation from wood-based biomass. The generated monosaccharides can be used not only for generation of bioethanol but also for other uses.

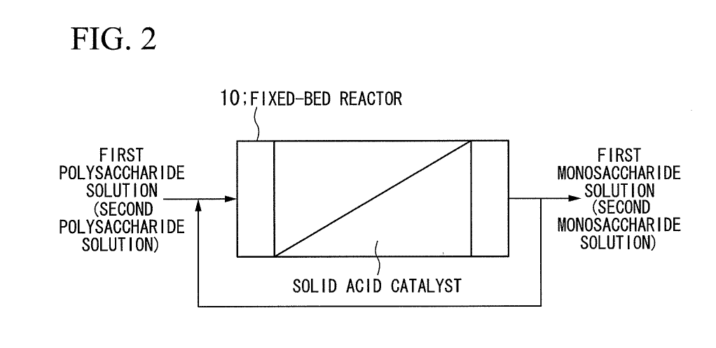

(2) In the embodiment, the first catalysis apparatus 2 constituted by the first mixing apparatus 2a and the first solid-liquid separating apparatus 2b is exemplified, but an embodiment may be a first catalysis apparatus in which a solid acid catalyst is fixed and filled in a fixed-bed reactor 10, the first polysaccharide solution including xylooligosaccharides is made to flow through the fixed-bed reactor so as to bring the first polysaccharide solution and the solid acid catalyst into contact and cause a saccharification reaction, thereby producing a liquid that flows out from the fixed-bed reactor 10 as the first monosaccharide solution including xylose as shown in FIG. 2. In this case, it is possible to produce the first monosaccharide solution having a higher purity by resending the liquid that flows out from the exit of the fixed-bed tank 10 to the entrance. Meanwhile, the above description equally applies to the second catalysis apparatus 3.

(3) In the embodiment, wood-based biomass, such as wood chips or construction waste, is used as a raw material; however, additionally, residue of crops or food (rice straw, wheat straw, bagasse, fruit skin, and the like) can be used as raw material biomass. In addition, the first reaction conditions and the second reaction conditions are set by combining the ratio K of the supply amount Q of pressurized hot water to the supply amount V of wood-based biomass and the temperature T of pressurized hot water in the embodiment, but the first reaction conditions and the second reaction conditions are not limited thereto, and may be set by combining the temperature T and residence time of pressurized hot water.

### INDUSTRIAL APPLICABILITY

According to the invention, it is possible to provide a biomass treatment apparatus and a method which can further improve the saccharide yield, the performance of a solid acid catalyst (specifically, improvement of the reaction rate), and ease of isolation.

### REFERENCE SIGNS LIST

A: BIOMASS TREATMENT APPARATUS; 1: PRESSURIZED HOT WATER REACTION APPARATUS; 2: FIRST CATALYSIS APPARATUS; 3: SECOND CATALYSIS APPARATUS; 4: FIRST FERMENTATION APPARATUS; 5: SECOND FERMENTATION APPARATUS; 6: DISTILLATION APPARATUS; 7: DRAINAGE TREATMENT APPARATUS

1. A biomass treatment apparatus comprising:
a pressurized hot water reaction apparatus in which pressurized hot water is used, biomass is hydrolyzed under first reaction conditions that provide for decomposition of hemicellulose so as to generate a first polysaccharide solution including xylooligosaccharides, and then the biomass is hydrolyzed under second reaction conditions that provide for decomposition of cellulose so as to generate a second polysaccharide solution including cellooligosaccharides;
a first catalysis apparatus in which the first polysaccharide solution that flows out from the pressurized hot water reaction apparatus is hydrolyzed using a solid acid catalyst so as to generate a first monosaccharide solution including xylose; and
a second catalysis apparatus in which the second polysaccharide solution that flows out from the pressurized hot water reaction apparatus is hydrolyzed using a solid acid catalyst so as to generate a second monosaccharide solution including glucose.

2. The biomass treatment apparatus according to claim 1,
wherein the first reaction conditions and the second reaction conditions are set by combining the ratio of the supply amount of the pressurized hot water and the supply amount of the biomass and the temperature of the pressurized hot water.

3. The biomass treatment apparatus according to claim 1,
wherein the first catalysis apparatus has:
a first mixing apparatus in which the first polysaccharide solution that flows out from the pressurized hot water reaction apparatus and the solid acid catalyst are mixed so as to generate the xylose; and
a first solid and liquid separating apparatus that separates the xylose that flows out from the first mixing apparatus and a first liquid mixture including the solid acid catalyst into solid and liquid so as to separate the first monosaccharide solution including the xylose and the solid acid catalyst, and collects and supplies the solid acid catalyst to the first mixing apparatus.

4. The biomass treatment apparatus according to claim 1,
wherein the second catalysis apparatus has:
a second mixing apparatus in which the second polysaccharide solution that flows out from the pressurized hot water reaction apparatus and the solid acid catalyst are mixed so as to generate the glucose; and
a second solid and liquid separating apparatus that separates the glucose that flows out from the second mixing apparatus and a second liquid mixture including the solid acid catalyst into solids and liquids so as to separate the second monosaccharide solution including the glucose and the solid acid catalyst, and, consequently, collects and supplies the solid acid catalyst to the second mixing apparatus.

5. The biomass treatment apparatus according to claim 1 further comprising:
a first fermentation apparatus that ferments the first monosaccharide solution generated by the first catalysis apparatus with an alcohol so as to generate bioethanol.

6. The biomass treatment apparatus according to claim 1 further comprising:
a second fermentation apparatus that ferments the second monosaccharide solution generated by the second catalysis apparatus with an alcohol so as to generate bioethanol.

7. The biomass treatment apparatus according to claim 1, wherein the first catalysis apparatus and the second catalysis apparatus are both connected to the pressurized hot water reaction apparatus separately.

8. A biomass treatment method comprising:
a pressurized hot water reaction process in which pressurized hot water is used, biomass is hydrolyzed under first reaction conditions that provide for decomposition of hemicellulose so as to generate a first polysaccharide solution including xylooligosaccharides, and then the biomass is hydrolyzed under second reaction conditions that provide for decomposition of cellulose so as to generate a second polysaccharide solution including cellooligosaccharides;
a first catalyst reaction process in which the first polysaccharide solution generated by the pressurized hot water reaction process is hydrolyzed using a solid acid catalyst so as to generate a first monosaccharide solution including xylose; and
a second catalyst reaction process in which the second polysaccharide solution generated by the pressurized hot water reaction process is hydrolyzed using a solid acid catalyst so as to generate a second monosaccharide solution including glucose.

9. The biomass treatment apparatus according to claim 2,
wherein the first catalysis apparatus has:
a first mixing apparatus in which the first polysaccharide solution that flows out from the pressurized hot water reaction apparatus and the solid acid catalyst are mixed so as to generate the xylose; and
a first solid and liquid separating apparatus that separates the xylose that flows out from the first mixing apparatus and a first liquid mixture including the solid acid catalyst into solid and liquid so as to separate the first monosaccharide solution including the xylose and the solid acid catalyst, and collects and supplies the solid acid catalyst to the first mixing apparatus.
